(19) United States
(12) Patent Application Publication
(10) Pub. No.: US 2021/0338218 A1
(43) Pub. Date: Nov. 4, 2021

(54) SYSTEMS AND METHODS FOR SEALING CORED OR PUNCTURED TISSUE

(71) Applicant: Ethicon, Inc., Somerville, NJ (US)

(72) Inventors: William Cohn, Houston, TX (US); Terry Daglow, Houston, TX (US); Matthew Kuhn, Houston, TX (US); Theodore Mick, Houston, TX (US); Chris Mills, Houston, TX (US); Steven Nguyen, Cyprus, TX (US); Tushar Sharma, Houston, TX (US)

(21) Appl. No.: 17/201,122

(22) Filed: Mar. 15, 2021

Related U.S. Application Data
(60) Provisional application No. 63/017,736, filed on Apr. 30, 2020.

(51) Int. Cl. A61B 17/00; A61B 18/18

(52) U.S. Cl. CPC A61B 17/0057; A61B 2018/0022; A61B 18/1815

(57) ABSTRACT

Systems and methods for sealing tissue sites may comprise coring tissue at a target site such that a tissue core is removed from the target site thereby creating a core cavity at the target site and causing sealing of at least a portion of the target site.

### CROSS-REFERENCE TO RELATED APPLICATIONS

This application claims priority to and the benefit of U.S. Patent Application No. 63/017,736 filed Apr. 30, 2020, which is hereby incorporated by reference in their entirety.

### BACKGROUND

Tissue, such as lung tissue, may become punctured or may have a portion removed. Removal of tissue may include a surgery that involves coring and removal of a tissue specimen, for example, from the lung. However, problems may arise with cored or punctured tissue. Improvements are needed in managing cored or punctured tissue, such as lung tissue.

### SUMMARY

It may be desirable to remove a core of tissue from target tissue sites including, but not limited to, the lungs, the liver, pancreas, or gastrointestinal (GI) tract, for which managing post-coring bleeding may be desired. A core of tissue may have a prescribed (e.g., pre-defined) shape (e.g., columnar) and dimensions based on a coring apparatus. Such coring apparatus may be used to core the same or substantially the same shaped tissue core in a repeatable manner. Such coring may be distinguished from other tissue removal, for example, using scissors or a scalpel, where the cut tissue will not have a pre-defined shape or dimensions.

Once cored, it may be desirable to seal the cored tissue. As described herein, sealing may be desirable during a coring process, for example, to seal blood and/or fluid flow at the core site that may be caused from the coring operation itself. Additionally or alternatively, sealing may be desirable after a core of tissue and/or a coring device is removed. Moreover, sealing may occur at least once during a coring process and at least once after a core of tissue and/or a coring device is removed. As an illustrative example, the cored site may be sealed during a coring process to limit undesired blood or fluids from entering the cored site. Then, the cored site may be sealed after the tissue core is removed to limit undesired air to escape through the cored site, such as in the lungs. Various sealing operations may be used.

Systems and/or methods for sealing tissue are described herein. A method for sealing tissue at a cored site may comprise coring tissue at a target site such that a tissue core is removed from the target site, thereby creating a core cavity at the target site. A sealing device may be disposed adjacent the target site. The sealing device may be caused to seal at least a portion of the core cavity at the target site. The sealing device may be spaced (e.g., removed, separated, etc.) from the target site.

Systems and/or methods for sealing tissue are described herein. An example method may comprise disposing a port to provide access to a target site. Example methods may comprise anchoring an anchor device, via the port, to a surface at the target site. Example methods may comprise disposing, via the port, a sealing device adjacent the target site. Example methods may comprise causing the sealing device to seal the target site. Example methods may comprise disposing a fill material adjacent the target site. Example methods may comprise minimizing, via the sealing device, escape of the fill material from the target site.

Systems and/or methods for sealing tissue are described herein. An example method may comprise disposing a sealing device adjacent a target site of a lung, while the lung is collapsed. Example methods may comprise causing the sealing device to seal the target site. Example methods may comprise disposing a fill material adjacent the target site. Example methods may comprise minimizing, via the sealing device, escape of the fill material from the target site. Example methods may comprise spacing (e.g., removing, separating, etc.) the sealing device from the target site.

Systems and/or methods for sealing tissue are described herein. An example method may comprise anchoring an anchor device to a surface at a target site. Example methods may comprise disposing, using the anchoring, a sealing device adjacent the target site. Example methods may comprise causing the sealing device to seal the target site. Example methods may comprise disposing a fill material adjacent the target site. Example methods may comprise minimizing, via the sealing device, escape of the fill material from the target site.

Systems and/or methods for sealing tissue are described herein. An example method may comprise disposing a fluid delivery device into a target site of a lung. Example methods may comprise disposing a fill material into the target site. Example methods may comprise spacing (e.g., removing, separating, etc.) the fluid delivery device from the target site.

### BRIEF DESCRIPTION OF THE DRAWINGS

The following drawings show generally, by way of example, but not by way of limitation, various examples discussed in the present disclosure. In the drawings:

FIG. 1 shows an application of an example system for sealing tissue.

FIG. 2 shows an application of an example system for sealing tissue.

FIG. 3 shows a flow diagram of an example method for sealing tissue.

FIG. 4 shows a flow diagram of an example method for sealing tissue.

FIG. 5 shows a flow diagram of an example method for sealing tissue.

FIG. 6 shows a flow diagram of an example method for sealing tissue.

FIG. 7 shows a flow diagram of an example method for coring and for sealing tissue.

FIGS. 8A, 8B, and 8C show an application of an example system for sealing tissue.

FIGS. 9A and 9B show an application of an example system for sealing tissue.

FIGS. 10A, 10B, and 10C show an application of an example system for sealing tissue.

FIG. 11 depicts a tissue resection device in accordance with an embodiment of the present disclosure.

FIG. 12 illustrates a sectional view of the tissue resection device of FIG. 1.

FIG. 13 shows a sectional view of a tissue resection device in accordance with an embodiment of the present disclosure.

FIG. 14 depicts a sectional view of a tissue resection device in accordance with an embodiment of the present disclosure.

FIG. 15 illustrates an exemplary anchor that may be employed in a lesion removal method in accordance with an embodiment of the present disclosure.

FIG. 16 shows a series of incision blades for use in a lesion removal method in accordance with an embodiment of the present disclosure.

FIG. 17 displays tissue dilators suitable for use in a lesion removal method in accordance with an embodiment of the present disclosure.

FIG. 18 shows a flow diagram of an example method for coring and for sealing tissue.

### DETAILED DESCRIPTION

The present disclosure relates to systems and methods for treatment of tissue such as cored tissue, punctured tissue, and/or a removed tissue region. Other tissue and sites may benefit from the disclosed systems and methods.

The present disclosure relates to methods and systems for coring tissue and sealing the core cavity created by removing the tissue core. Such methods may comprise disposing a fill material in the core cavity. Methods may comprise applying pressure to a portion of the core cavity such as to a wall defining the core cavity. Methods may comprise ablating a portion of the core cavity such as a wall defining the core cavity. Methods may comprise causing a cavity closure device, such as suture thread, a stapling device, an ultrasonic tissue sealing device, a bipolar radiofrequency sealing device, or any combination thereof to close the tissue cavity. Methods may comprise disposing a cavity sealing material, such as a tissue graft, a hemostatic patch, a hemostatic agent such as fibrin or thrombin, a biological adhesive material such as Dermabond®, or any combination thereof to close the tissue cavity.

Methods may comprise any combination or permutation of: 1) disposing an anchoring device into a tissue cavity, 2) disposing a tissue access port into the tissue cavity, 3) disposing a tissue sealing device into the tissue cavity (with or without a tissue access port, with or without guidance from an anchoring device), 4) causing the tissue sealing device to seal at least a portion of the tissue cavity, 5) introducing a fill material into the tissue cavity (with or without a fill material delivery device, with or without being preceded by disposing a tissue sealing device into the tissue cavity, with or without removing the tissue sealing device after sealing at least a portion of the tissue cavity, with or without a tissue access port), 6) disposing a cavity sealing material adjacent to the tissue cavity (with or without being preceded by disposing a tissue sealing device into the tissue cavity, with or without removing the tissue sealing device after sealing at least a portion of the tissue cavity, with or without being preceded by introducing a fill material into the tissue cavity), 7) disposing a cavity closure device adjacent to the tissue, and 8) causing a cavity closure device to close the tissue cavity (with or without being preceded by any combination or permutation of the above steps). As described herein, methods may be used to core and/or seal tissue at various target sites. Although a lung is used as an illustrative example, it should not be so limiting, as other target sites may be punctured or actively cored and may benefit from the disclosed sealing methods.

The present disclosure relates to a method to deliver a fill material such as autologous blood to the core site that may be used to seal and provide pneumostasis. As an example, once the tissue specimen is cored and removed from the lung, there may be a need to seal the core site to provide pneumostasis. As a further example, pneumostasis may be achieved in the same surgery session as the tissue removal.

Although autologous blood is described herein as an example, other fill materials and additives may be used. For example, a hemostatic adjunct such as an absorbable gelatin foam (e.g., SURGIFOAM®), biologic, oxidized regenerated cellulose (ORC), fibrin/thrombin spray, etc. As a further example, a patient may have a rare disorder of hemophilia in which their blood does not clot normally. Other patients might be on blood thinning medicines which could inhibit blood clotting formation. For such patients, to seal the cored cavity, thrombin and/or fibrinogen may be added to the autologous blood sample to aid in clot formation. Reactive polyethylene glycol (PEG), ammonium sulfate, ethanol, calcium chloride, or magnesium chloride may also be added to the blood sample to aid in clot formation. Another source for the blood to be used to seal the cored cavity is donated blood from other people or a blood bank. Donated blood may be used with or without clotting agents as mentioned above.

Systems and/or methods for sealing tissue are described herein. An example method may comprise disposing a port to provide access to a target site. The target site may comprise biological tissue. The target site may comprise tissue of a lung. The target site may comprise a cored tissue. The target site may comprise a punctured tissue. Other sites may benefit from the disclosed methods.

Example methods may comprise anchoring an anchor device (e.g., via the port) to a surface at the target site. Anchoring may be performed by any suitable structure for securing the device to the lung. Example methods may comprise disposing (e.g., via the port) a sealing device adjacent the target site. Example methods may comprise disposing a sealing device adjacent the target site using the anchoring device as a guide. The sealing device may comprise an inflatable balloon. The sealing device may comprise an inflatable balloon with an array of radio frequency (RF) electrodes configured to ablate and seal tissue. The sealing device may comprise an inflatable balloon configured to seal tissue using a thermal fluid. The sealing device may comprise an inflatable balloon catheter. The sealing device may comprise an access port with an array of RF electrodes configured to ablate and seal tissue. The sealing device may comprise at least one microwave ablation probe.

Example methods may comprise causing the sealing device to seal the target site. The causing the sealing device to seal the target site may comprise causing at least a portion of the sealing device to abut a portion of the target site. Example methods may comprise disposing a fill material adjacent the target site. Example methods may comprise disposing a fill material adjacent the target site via a fill material delivery device such as a catheter. The fill material may comprise autologous blood, donated blood, recirculated blood, hemostatic adjuncts such as fibrin and/or thrombin, biological tissue adhesives such as Dermabond®, ORC, absorbable gelatin, or any combination thereof. The fill material may promote pneumostasis. The fill material may additionally promote hemostasis. Other materials may be used. The sealing device may minimize escape of the fill material from the target site.

As an illustrative example, the target site may comprise at least a portion of a lung. The lung may be caused to collapse prior to disposing the sealing device adjacent the target site. The lung may be allowed to ventilate while the sealing device is sealing the target site. The sealing device may be spaced (e.g., removed, separated, etc.) from the target site after the fill material is disposed.

Systems and/or methods for sealing are described herein. An example method may comprise disposing a sealing device adjacent a target site of a lung. The sealing device may be disposed adjacent the target site while the lung is collapsed. However, the lung may be ventilated. Example methods may comprise causing the sealing device to seal the target site. Example methods may comprise disposing a sealing device adjacent the target site using the anchoring device as a guide. The sealing device may comprise an inflatable balloon. The sealing device may comprise an inflatable balloon with an array of RF electrodes configured to ablate and seal tissue. The sealing device may comprise an inflatable balloon configured to seal tissue using a thermal fluid. The sealing device may comprise an inflatable balloon catheter. The sealing device may comprise an access port with an array of RF electrodes configured to ablate and seal tissue. The sealing device may comprise at least one microwave ablation probe. Example methods may comprise disposing a fill material adjacent the target site. Example methods may comprise disposing a fill material adjacent the target site via a fill material delivery device such as a catheter. The fill material may comprise autologous blood, donated blood, recirculated blood, hemostatic adjuncts such as fibrin and/or thrombin, biological tissue adhesives such as Dermabond®, ORC, absorbable gelatin, or any combination thereof. The fill material may promote pneumostasis. The fill material may additionally promote hemostasis. Other materials may be used. The sealing device may minimize escape of the fill material from the target site.

Systems and/or methods for sealing are described herein. An example method may comprise disposing a fluid delivery device into a target site of a lung. The sealing device may be disposed adjacent the target site, while the lung is collapsed. Alternatively, the lung may be ventilated. Example methods may comprise disposing a fill material into the target site. Example methods may comprise spacing (e.g., removing, separating, etc.) the sealing device from the target site.

The sealing device may comprise an inflatable balloon. The sealing device may comprise an inflatable balloon with an array of RF electrodes configured to ablate and seal tissue. The sealing device may comprise an inflatable balloon configured to seal tissue using a thermal fluid. The sealing device may comprise an inflatable balloon catheter. The sealing device may comprise an access port with an array of RF electrodes configured to ablate and seal tissue. The sealing device may comprise at least one microwave ablation probe. The systems and/or methods described herein may allow clotted blood to provide a seal to achieve pneumostasis. Example methods may comprise disposing a fill material adjacent the target site. Example methods may comprise disposing a fill material adjacent the target site via a fill material delivery device such as a catheter. The fill material may comprise autologous blood, donated blood, recirculated blood, hemostatic adjuncts such as fibrin and/or thrombin, biological tissue adhesives such as Dermabond®, ORC, absorbable gelatin, or any combination thereof. The fill material may promote pneumostasis. The fill material may additionally promote hemostasis. Other materials may be used. The sealing device may minimize escape of the fill material from the target site.

The target site may comprise a cavity. The cavity may be closed, for example, after sealing. Closing the cavity may comprise using biological tissue adhesive such as Dermabond®, tissue grafts, hemostatic sealing patches, staple closure, sutures, or the like.

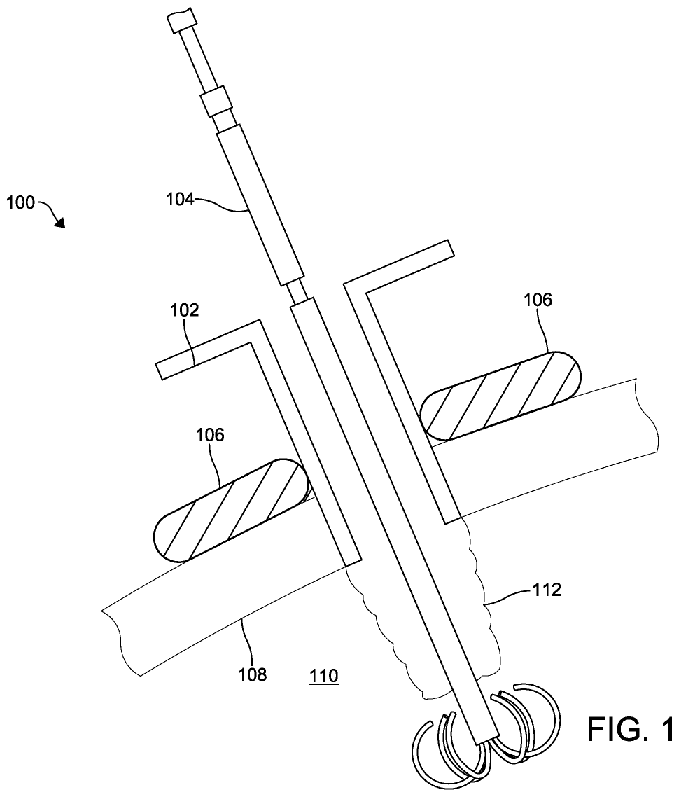

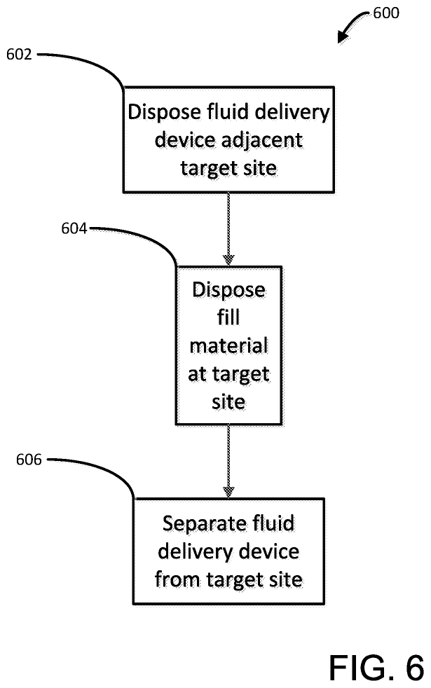

FIG. 1 shows an example system 100. The system 100 may comprise a port such as chest port 102 configured to provide access, such as via a channel to a portion of a body. It should be understood that various channels or ports may be used throughout the body and the chest port 102 is shown as a non-limiting example. As an illustrative example, the chest port 102 is shown disposed adjacent ribs 106 to provide access to a lung 110 of a patient. However, other sites may be used and a chest port 102 (or other port) may not be necessary. An anchor device 104 may be anchored to tissue, such as the lung 110. An example anchor device is shown in FIG. 6 for illustration. However, any suitable device for anchoring to the target site 112 may be used. As show, the anchor device 104 extends via the chest port 102, through the pleura 108, and anchors to tissue in the lung 110. The anchor device 104 may be anchored (e.g., releasably coupled) to a tissue at a target site 112. The target site 112 may comprise a core site where a portion of lung tissue has been cored, punctured, or removed. The anchor device 104 may be placed at the target site 112 while the lung is inflated. However, other processes may be implemented while the lung is collapsed.

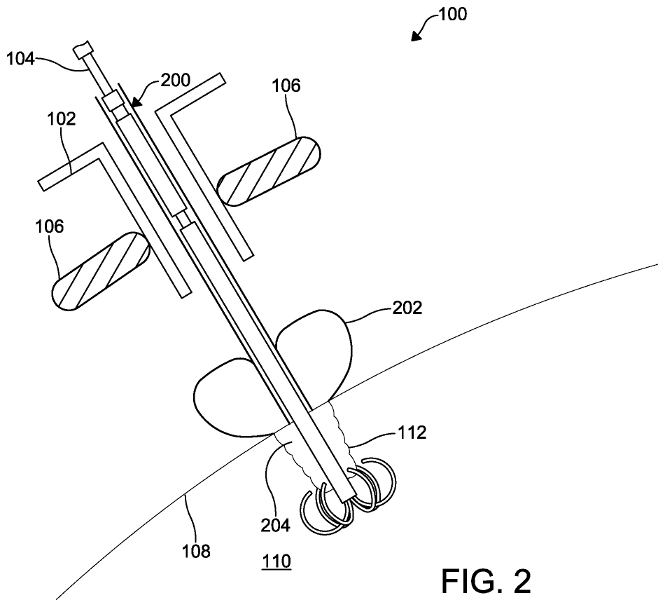

FIG. 2 shows an application of an example sealing device 200. The sealing device 200 may comprise an inflatable balloon 202. Other sealing mechanisms may be used. The sealing device 200 may comprise and/or be in contact with a balloon catheter. The balloon catheter may be a single lumen balloon catheter. The balloon catheter may be a multi-lumen balloon catheter. The sealing device 200 may be disposed adjacent the target site 112. As such, the sealing device 200 may seal the target site 112 to minimize exit of a fluid or material from the target site 112. As an example, a fill material 204 may be disposed at the target site 112 and may be sealed in the target site 112 by the sealing device 200. As an illustrative example, the inflatable balloon 202 may provide sealing while the lung 110 moves (e.g., inflates and deflates). The sealing device 200 may be implemented when the lung 110 is inflated or collapsed.

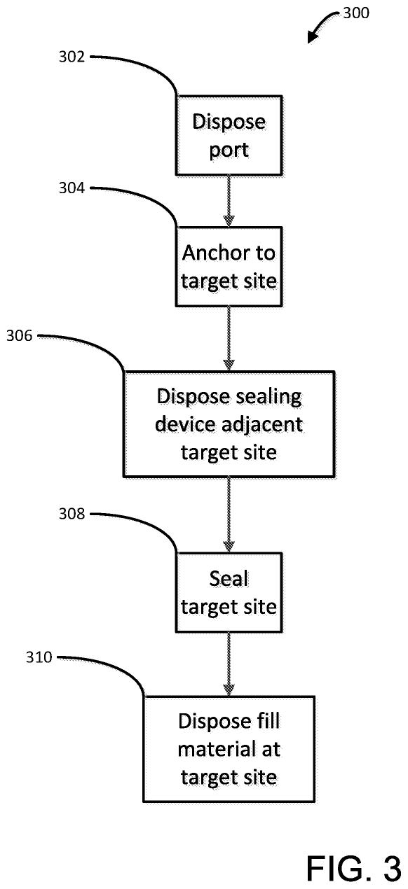

FIG. 3 shows a flow diagram of an example method 300. At 302, a port may be disposed to provide access to a target site. A port (e.g., chest port 102 (FIG. 1)) may be disposed to provide access to a target site. A user may dispose a port to provide access to a target site. AS an example, a port may be disposed adjacent the target site. The target site may comprise biological tissue. The target site may comprise tissue of a lung. The target site may comprise a cored tissue. The target site may comprise a punctured tissue. The target site may comprise at least a portion of a lung.

At 304, an anchor device may be anchored to a surface at the target site. As an example, a user may anchor the anchor device, via the port, to a surface at the target site.

At 306, a sealing device may be disposed adjacent the target site, for example via the port. The sealing device may comprise an inflatable balloon. The sealing device may comprise an inflatable balloon catheter. The lung may be caused to collapse prior to disposing the sealing device adjacent the target site. The lung may be allowed to ventilate while the sealing device is sealing the target site.

At 308, the sealing device may be caused to seal the target site. Causing the sealing device to seal the target site may comprise causing at least a portion of the sealing device to abut a portion of the target site.

At 310, a fill material may be disposed adjacent the target site. The sealing device may minimize escape of the fill material from the target site. The fill material may promote pneumostasis. The fill material may comprise autologous blood. The sealing device may be spaced (e.g., removed, separated, etc.) from the target site after the fill material is disposed.

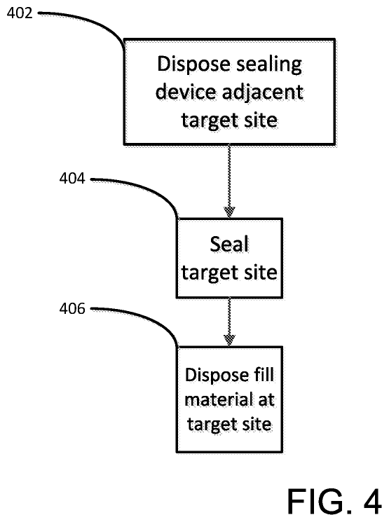

FIG. 4 shows a flow diagram of an example method 400. At 402, a sealing device may be disposed adjacent a target site of a lung, while the lung is collapsed. The target site may comprise tissue of a lung. The target site may comprise a cored tissue. The target site may comprise a punctured tissue. The sealing device may comprise an inflatable balloon. The sealing device may comprise an inflatable balloon catheter.

At 404, the sealing device may be caused to seal the target site. Causing the sealing device to seal the target site may comprise causing at least a portion of the sealing device to abut a portion of the target site.

At 406, a fill material may be disposed adjacent the target site. The sealing device may minimize escape of the fill material from the target site. The fill material may promote pneumostasis. The fill material may comprise autologous blood. At 408, the sealing device may be spaced (e.g., removed, separated, etc.) from the target site.

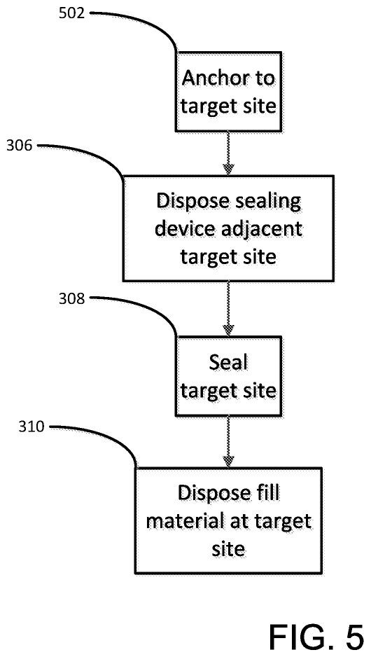

FIG. 5 shows a flow diagram of an example method 500. At 502, an anchor device may be anchored to a surface at a target site. The target site may comprise biological tissue. The target site may comprise tissue of a lung. The target site may comprise a cored tissue. The target site may comprise a punctured tissue. The target site may comprise at least a portion of a lung.

At 504, a sealing device may be disposed adjacent the target site using the anchoring. The sealing device may comprise an inflatable balloon. The sealing device may comprise an inflatable balloon catheter. The lung may be caused to collapse prior to disposing the sealing device adjacent the target site.

At 506, the sealing device may be caused to seal the target site. Causing the sealing device to seal the target site may comprise causing at least a portion of the sealing device to abut a portion of the target site. The lung may be allowed to ventilate while the sealing device is sealing the target site.

At 508, a fill material may be disposed adjacent the target site. The sealing device may minimize escape of the fill material from the target site. The fill material may promote pneumostasis. The fill material may comprise autologous blood. The sealing device may be spaced (e.g., removed, separated, etc.) from the target site after the fill material is disposed.

FIG. 6 shows a flow diagram of an example method 600. At 602, a fluid delivery device may be disposed adjacent or into a target site of a lung, while the lung is collapsed. The target site may comprise biological tissue. The target site may comprise tissue of a lung. The target site may comprise cored tissue. The target site may comprise punctured tissue. The target site may comprise at least a portion of a lung.

At 604, a fill material may be disposed into the target site. The fill material may promote pneumostasis. The fill material may comprise autologous blood.

At 606, the fluid delivery device may be spaced (e.g., removed, separated, etc.) from the target site. The lung may be caused to collapse prior to disposing the fluid delivery device adjacent the target site.

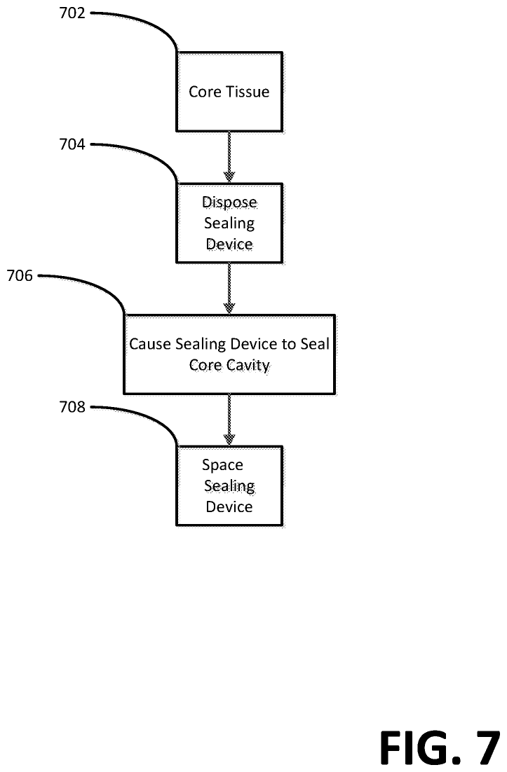

FIG. 7 shows a flow diagram of an example method. At 702, tissue at a target site may be cored such that a tissue core is removed from the target site thereby creating a core cavity at the target site. Coring tissue at a target site may comprise transecting and sealing tissue. Coring tissue at a target site may comprise disposing a tissue coring apparatus adjacent to a target tissue site. The tissue coring apparatus may comprise a first clamping element comprising a helical coil, a second clamping element, the second clamping element being positioned to oppose at least a portion of the first clamping element, a first and second electrode configured for the delivery of radiofrequency energy for sealing tissue, and/or a cutting element configured for the transection of at least a portion of the sealed tissue. Other apparatus may be used.

Coring tissue at a target site may comprise using the tissue coring apparatus to form the tissue core. The target site may be a tissue site such as a lung, for example. At 704, a sealing device may be disposed adjacent the target site. The sealing device may comprise an inflatable balloon. The sealing device may comprise an inflatable balloon catheter. At 706, the sealing device may be caused to seal at least a portion of the core cavity at the target site. Example sealing procedures are described herein and include fill materials, ablation, mechanical pressure, energy emission (e.g., RF energy), and others, for example. Causing the sealing device to seal at least a portion of the core cavity at the target site may comprise causing at least a portion of the sealing device to abut a wall defining the core cavity. Causing the sealing device to seal at least a portion of the core cavity at the target site may comprise ablating a wall defining the core cavity. Causing the sealing device to seal at least a portion of the core cavity at the target site may comprise applying pressure to a wall defining the core cavity. The method 700 may further comprise disposing a fill material in the core cavity, wherein the sealing device minimizes escape of the fill material from the core cavity. The fill material may comprise autologous blood. As an example, the target site may comprise at least a portion of a lung and the method may further comprise causing the lung to collapse prior to disposing the sealing device adjacent the target site. As a further example, the target site may comprise at least a portion of a lung and the method 700 may further comprise allowing the lung to ventilate while the sealing device is sealing the target site. At 708, the sealing device may be spaced (e.g., removed, separated, etc.) from the target site.

An example system for implementing one or more of the methods of the present disclosure may comprise a guided anchor. The example system may comprise a single lumen balloon catheter. The example system may comprise a multi-lumen balloon catheter. The example system may comprise a coring device. Post coring by the coring device, an anchor may be introduced into the tissue cavity to ensure access to a cored site, as shown in FIG. 1. The chest port may be removed, and the lung may be collapsed. The balloon catheter may be inserted over the anchor. Once the balloon catheter is in the chest cavity, the balloon catheter may be inflated. The inflated balloon catheter may be moved forward and pushed slightly against lung tissue. Autologous blood may be injected into a core site through the inflated balloon catheter. The inflated balloon catheter and autologous blood may be held in place for a predetermined time period (e.g., one (1) minute, etc.) to allow the blood to clot at the core site, as shown in FIG. 2. The lung may be allowed to resume ventilation. The inflated balloon catheter may be allowed to go up and down with the lung while maintaining contact with the lung to keep the blood at the core site to facilitate further clotting. The balloon catheter may be deflated. The balloon catheter and anchor may be removed after a predetermined time period (e.g., three (3) minutes, etc.). The autologous blood may be clotted at the core site to provide pneumostasis.

In an embodiment, the anchor and/or the balloon catheter may be used to deposit autologous blood at the core site with the lung collapsed. The anchor and/or the balloon catheter may be removed right after the autologous blood is delivered. The blood may be allowed to clot in place with a predetermined time period (e.g., five (5) minutes, etc.) before the lung is allowed to resume ventilation.

The example system may cause autologous blood to be delivered to the core site. Other fill materials may be used.

The example system may allow clotted blood to provide a seal to achieve pneumostasis.

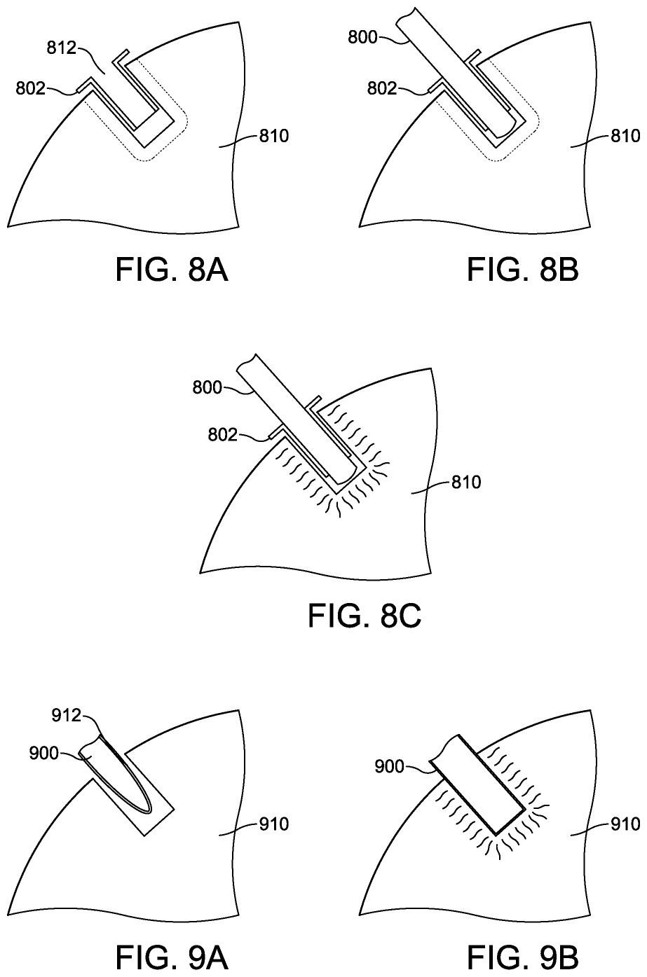

FIGS. 8A-8C show an example application. As shown, once a target site has been cored out and the tissue core removed, there may be a need to ablate the tissue wall of the cavity. As such, the following ablation methods could be used. For example, a rotating ablation probe may be used. FIG. 8A shows a cored-out cavity 812 in tissue 810 with the cavity sheath 802 in place to keep the cavity open. A rotating probe 800 is then inserted into the cavity sheath as shown in FIG. 8B. The probe 800 may be equipped with an energy source such as an array of energy heads or a continuous energy strip. The energy may be microwave, RF, other output form. Once the probe 800 is in place, the cavity sheath 802 may remain in place or be removed. The energy is then applied while the probe/energy heads are rotated to give a radially continuous ablation on the wall and bottom tissue 810 of the cavity as shown in FIG. 8C.

FIGS. 9A-9B show an example application. As shown, a hot air balloon catheter may be used. For example, a balloon catheter 900 may be placed into a cavity 912 formed in tissue 910 and a cavity sheath may be removed to expose the cavity 912 needing to be ablated, as shown in FIG. 9A. The balloon 900 may then be inflated with hot fluid or hot air/gas to ablate the cavity wall tissue 910, as shown in FIG. 9B.

FIGS. 8-9 show illustrative examples, but other methods of ablation or energy emission may be used for sealing tissue. For example, a shaped mesh catheter may be used. As such, a catheter with collapsed meshed shape may be inserted into the cavity and the cavity sheath is removed. The mesh may then be expanded, and suction may be applied to pull tissue to contact with the mesh. Energy, e.g. RF energy, may then be applied to ablate the cavity tissue wall.

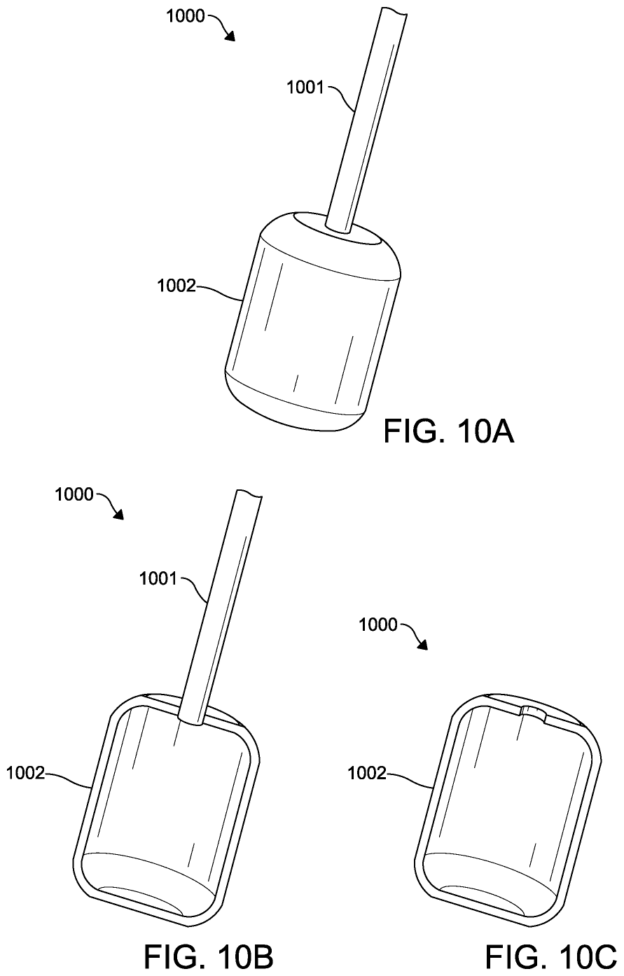

FIGS. 10A-10C show an example application. As shown, once a target site has been cored out and the tissue core removed, there may be a need to seal the cut tissue wall of the cavity. As such, the following example procedure may be used. A device 1000 may comprise a fluid conduit 1001 and an inflatable absorbable balloon 1002. The balloon 1002 may be coated on the exterior with absorbable bio adhesive that will seal against the tissue of the cored cavity post coring, as shown in FIGS. 10A-10B. Once the deflated balloon 1002 may be placed in the desired location, the balloon 1002 may be inflated with CO2 (or other fluid), for example via fluid conduit 1001, so that the bio adhesive presses against the tissue wall of the cored cavity to achieve sealing to prevent air leak. The CO2 filled balloon 1002 may be pressurized to an appropriate pressure and may be left behind inside the cored cavity, as shown in FIG. 10C.

Various methods, devices, and systems may be used to core or remove tissue.

A method for removing a tissue lesion may comprise introducing a tissue resection device to a target tissue site, causing the tissue resection device to resect a core of tissue from the target tissue site, and removing the core of tissue from the body. The core of tissue may comprise at least a portion of a tissue lesion. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for the delivery of radiofrequency energy. The tissue resection device may be configured for mechanical transection. The tissue resection device may comprise mechanical compression and the delivery of radiofrequency energy. A method may further comprise amputating the core of tissue from the target tissue site. As an example, the means for amputation of the core of tissue may comprise mechanical transection. As a further example, the means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire. Other devices may be used.

A method for removing a core of tissue may comprise introducing a tissue resection device to a target tissue site, causing the tissue resection device to resect a core of tissue from the target tissue site, and removing the core of tissue from the body. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for the delivery of radiofrequency energy. The tissue resection device may be configured for mechanical transection. The tissue resection device may be configured for mechanical compression and the delivery of radiofrequency energy. A method may further comprise amputating the core of tissue from the target tissue site. The means for amputation of the core of tissue may comprise mechanical transection. The means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire.

A method for removing a core of tissue may comprise introducing a tissue resection device to a target tissue site. The tissue resection device may comprise one or more of: a first clamping element comprising a helical coil and a first electrode, or a second clamping element comprising a second electrode. Where a second clamping element is included, the second clamping element may be positioned to oppose at least a portion of the first clamping element. The method may further comprise causing the tissue resection device to resect a core of tissue from the target tissue site and removing the core of tissue from the body. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for resecting the core of tissue comprises the delivery of radiofrequency energy. The tissue resection device may be configured for resecting the core of tissue comprises mechanical transection. The tissue resection device may be configured for resecting the core of tissue comprises mechanical compression and the delivery of radiofrequency energy. A method may further comprise amputating the core of tissue from the target tissue site. The means for amputation of the core of tissue may comprise mechanical transection. The means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire.

A method for sealing biological fluid vessels may comprise piercing a target tissue site containing a least a portion of at least one target biological fluid vessel with a helical tissue sealing mechanism. The helical tissue sealing mechanism may comprise a helical piercing element and a clamping element. The method may comprise causing the helical tissue sealing mechanism to apply mechanical compression to at least one target biological fluid vessel and delivering energy to seal at least one target biological fluid vessel. The helical piercing element may comprise the clamping element. The mechanical compression may be applied between the helical piercing element and the clamping element. A method may further comprise a second clamping element. The mechanical compression may be applied between the first and second clamping elements. The delivered energy may comprise monopolar radiofrequency energy. The delivered energy may comprise bipolar radiofrequency energy. The delivered energy may comprise thermal energy. The delivered energy may comprise ultrasonic energy.

A method for sealing biological fluid vessels may comprise piercing a target tissue site with a helical piercing element, adjusting the pitch of the helical piercing element to apply mechanical compression to the target tissue, and delivering energy to seal at least one biological fluid vessel in the target tissue. The helical piercing element may comprise a plurality of tissue sealing electrodes. The delivered energy may comprise monopolar radiofrequency energy. The delivered energy may comprise bipolar radiofrequency energy. The delivered energy may comprise thermal energy. The delivered energy may comprise ultrasonic energy.

A tissue resection apparatus may comprise a first clamping element comprising a helical coil, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. A tissue resection device may further comprise a first actuator operable to actuate the first or second clamping element to apply mechanical compression to tissue and a second actuator operable to actuate the cutting element to transect tissue. The helical coil may include first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element having a helical coil disposed on a distal end, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. The tissue resection device may further comprise a first actuator operable to actuate the first or second clamping element to apply mechanical compression to tissue and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element comprising a helical coil and a first electrode, and a second clamping element comprising a second electrode, the second clamping element being positioned to oppose at least a portion of the first clamping element. The first and second clamping elements may be configured for: (a) the delivery of radiofrequency energy for sealing tissue, and (b) the application of mechanical compression for the transection of tissue. The tissue resection device may further comprise a first actuator operable to actuate the first or second clamping element to apply mechanical compression to tissue and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A surgical instrument system for the resection of tissue may comprise an end effector operable to cut and seal tissue, wherein the end effector and a generator configured to provide power to the end effector having the first and second electrodes for sealing tissue. The end effector may comprise a first clamping element comprising a helical coil, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. The surgical instrument system may further comprise a controller in communication with the generator, wherein the controller is configured to control the generator to provide radiofrequency energy sufficient to seal tissue to the first and second electrodes of the end effector, based on at least one sensed operating condition of the end effector. The controller may be configured to sense the presence of tissue at the end effector. The controller may be configured to sense the presence of tissue at the end effector based on a measured impedance level associated with the first and second electrodes. The controller may be configured to sense an amount of force applied to at least one of the first or second clamping elements to detect the presence of tissue at the end effector. The controller may be configured to sense the position of the cutting element relative to at least one of the first or second clamping elements. The controller may be configured to control the generator to provide radiofrequency energy at the end effector when the second actuator is actuated and no tissue is sensed at the end effector. The controller may be configured to control the generator to provide a continuous amount of radiofrequency energy. The controller may be configured to control the generator to automatically provide an increase or decrease in the amount of radiofrequency energy. The system may further comprise a first actuator operable to actuate the first or second clamping element to apply mechanical compression to tissue and a second actuator operable to actuate the cutting element to transect tissue. The helical coil may comprise first and second contiguous coil segments, the first coil segment including the first electrode. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element comprising a helical coil, a second clamping element, the second clamping element being positioned to oppose at least a portion of the first clamping element, a first and second electrode configured for the delivery of radiofrequency energy for sealing tissue, a first cutting element configured for the transection of at least a portion of the sealed tissue, a first and second ligating element, and a second cutting element positioned between said first and second ligating elements. The tissue resection device may further comprise a first actuator operable to actuate the first or second clamping element to apply mechanical compression to tissue, and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue sealing mechanism may comprise a helical coil with a generally obround cross section and a tapered point disposed at a distal end, a first and second helical tissue sealing surface, wherein the first and second helical tissue sealing surfaces are provided by the parallel planar surfaces of the helical coil, a first electrode disposed on the first helical tissue sealing surface, and a second electrode disposed on the second helical tissue sealing surface, wherein the first and second electrodes are configured to apply bipolar radiofrequency energy for sealing tissue. The helical coil may comprise first and second contiguous coil segments. The helical coil may comprise a blunt tip. The first and second electrodes may have surface profiles that are substantially matching. The first and second helical tissue sealing surfaces may further comprise a plurality of electrodes configured for the delivery of bipolar radiofrequency energy.

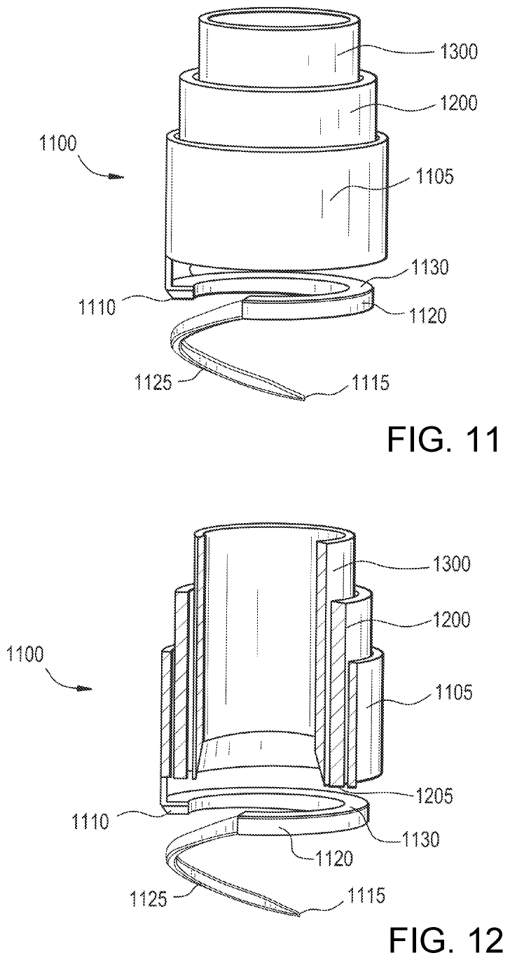

FIGS. 11-17 show example devices that may be used to effect a coring process, as described herein. For example, a resection device of the present invention may comprise an energy-based arrangement capable of penetrating tissue towards a target lesion. In one embodiment depicted in FIG. 11, a tissue resection device 1100 includes an outer tube 1105 is provided having a distal edge profile and having an inner diameter IDouter. A coil 1110 is attached to an outer tube 1105 where the coil turns are spaced from and opposed to a distal end of the outer tube 1105. The coil 1110 preferably has a slightly blunted tip 1115 to minimize the possibility that it will penetrate through a blood vessel while being sufficiently sharp to penetrate tissue such as pleura and parenchyma. In some embodiments, the coil 1110 may take the form of a helix having a constant or variable pitch. The coil 1110 may also have a variable cross-sectional geometry. An electrode 1130 may be disposed on a surface or embedded within the coil 1110.

In some embodiments, as illustrated in FIG. 11, the coil 1110 may include a plurality of contiguous coil segments, e.g., coil segments 1120 and 1125. The coil segment 1120 may comprise a helical member having a pitch of zero, e.g., a generally planar open ring structure, having an inner diameter IDcoil and an outer diameter ODcoil. The coil segment 1125 may comprise a helical structure of constant or variable pitch and constant or variable cross-sectional geometry. In this embodiment, the electrode 1130 may be disposed on a surface of or embedded in the coil segment 1120.

A central tube 1200 may be provided having a distal end with an edge profile comprising one or more surface segments and having an outer diameter ODcentral and an inner diameter IDcentral. As illustrated in FIG. 12, an electrode 1205 may be disposed on or embedded within at least one of the surface segments. A central tube 1200 may be slidably disposed within the outer tube 1105 and positioned such that electrode 1205 opposes and overlaps at least a portion of electrode 1130. The space between electrode 1205 and electrode 1130 may be referred to as the tissue clamping zone. In keeping with an aspect of the present disclosure, ODcentral>IDcoil and ODcoil>IDcentral. In some embodiments, ODcentral may be about equal to ODcoil. Accordingly, the central tube 1200 may be advanced through the tissue clamping zone towards the coil 1110 such that electrode 1205 abuts electrode 1130.

A cutting tube 1300 may be slidably disposed within the central tube 1200. The distal end of the cutting tube 1300 is provided with a knife edge to facilitate tissue cutting.

To enable tissue resection, the resection device 1100 may be inserted into tissue and the outer tube 1105 may be advanced a predetermined distance towards a target. The coil segment 1125 may allow the device to penetrate the tissue in a manner similar to a cork screw. As the coil segment 1125 penetrates tissue, any vessel in its path may either be moved to the planar coil segment 1120 or pushed away from the coil 1100 for subsequent turns. The coil tip 1115 may be made blunt enough to minimize the chance that it will penetrate through a blood vessel, while still sharp enough to penetrate certain tissue, such as the lung pleura and parenchyma. The central tube 1200 may then be advanced a predetermined distance towards the target. Any vessels that are disposed in the tissue clamping zone may be clamped between electrode 1130 and electrode 1205. The vessels may then be sealed by the application of bipolar energy to electrode 1130 and electrode 1205. Once the blood vessels are sealed, the cutting tube 1300 may advance to core the tissue to the depth that the outer tube 1105 has reached. The sealing and cutting process may be repeated to create a core of a desired size.

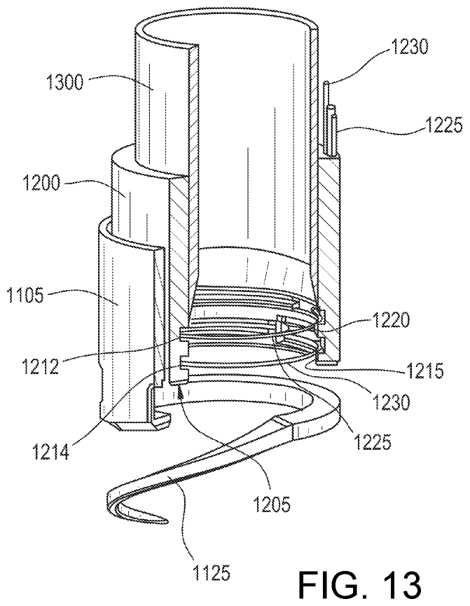

In keeping with an aspect of the present disclosure, the resection device may be further configured to dissect a target lesion and seal tissue proximate the dissection point. To facilitate dissection and sealing, as illustrated in FIG. 13, the central tube 1200 may be provided with a ligation snare 1230, first and second ligation electrodes 1215 and 1220, and an amputation snare 1225. As used herein, the word “snare” refers to a flexible line, e.g., a string or a wire. The inner wall surface of the central tube 1200 may include upper and lower circumferential grooved pathways 1212 and 1214 disposed proximate the distal end. The first and second ligation electrodes 1215 and 1220 may be disposed on the inner wall of central tube 1200 such that the lower circumferential groove 1214 may be between them. The upper grooved pathway 1212 may be disposed axially above the ligation electrodes 1215 and 1220.

The ligation snare 1230 may be disposed in the lower circumferential groove 1214 and extends through the central tube 1200 and axially along the outer wall surface to a snare activation mechanism (not shown). The amputation snare 1225 may be disposed in the upper circumferential groove 1212 and extend through the central tube 1200 and axially along the outer wall surface to a snare activation mechanism (not shown). The outer surface of the central tube 1200 may be provided with a plurality of axially extending grooved pathways which receive the amputation snare 1225 and the ligation snare 1230 and are in communication with the upper and lower circumferential grooved pathways 1212 and 1214. In addition, electrode leads for the ligation electrodes 1215 and 1220 may extend to an energy source via the axially extending grooved pathways.

In operation, the resection device of this embodiment may detach and seal the tissue core. The cutting tube 1300 may be retracted to expose the ligation snare 1230 which may preferably be made of flexible line, e.g., suture. The ligation snare 1230 may be engaged to snag tissue and pull tissue against the inner wall surface between the first and second ligation electrodes 1215 and 1220. Bipolar energy may then be applied to the first and second electrodes 1215 and 1220 to seal, i.e., cauterize, the tissue. Once sealed, the cutting tube 1300 may be further retracted to expose the amputation snare 1225, which may then be activated to sever the tissue core upstream from the point where the tissue was sealed (ligation point). In some embodiments, the amputation snare 1225 has a smaller diameter than that of the ligation snare 1230. The smaller diameter may facilitate tissue slicing. Accordingly, the resection device 1100 according to this embodiment may both create a tissue core and disengage the core from surrounding tissue.

In an alternative embodiment, the resection device of the present disclosure may be provided with a single snare disposed between ligation electrodes which both ligates and cuts tissue. In this embodiment, the single snare may first pull tissue against the inner wall surface of the central tube 1200 between the ligation electrodes 1215 and 1220. Bipolar energy may then then applied to first and second electrodes 1215 and 1220 to seal, i.e., cauterize, the tissue. Once sealed, the snare may be further pulled to sever the tissue core.

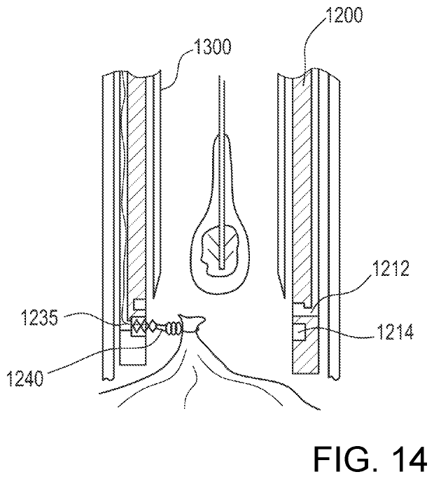

In yet another embodiment, cutting and sealing may be performed without employing electrodes. In this embodiment, the ligation snare 1230 may include a set of ligation knots 1235 and 1240 which tighten under load, shown, for example, in FIG. 14. Ligation may be performed by retracting the cutting tube 1300 to expose the ligation snare 1230 and activating the ligation snare 1230, which lassos tissue as one or more of the ligation knots 1235 and 1240 tightens. Once the tissue is lassoed, the cutting tube 1300 may be further retracted to expose the amputation snare 1225 which may then be activated to sever the tissue core upstream from the point where the point where the tissue was lassoed.

The present disclosure also contemplates a method and system for using the resection device to remove tissue lesions, for example, lung lesions. The method generally comprises anchoring the lesion targeted for removal, creating a channel in the tissue leading to the target lesion, creating a tissue core which includes the anchored lesion, ligating the tissue core and sealing the surrounding tissue, and removing the tissue core including the target lesion from the channel.

Anchoring may be performed by, any suitable structure for securing the device to the lung. Once the lesion is anchored, a channel may be created to facilitate insertion of the resection device 1100. The channel may be created by making an incision in the lung area and inserting a tissue dilator and port into the incision. A tissue core which includes the anchored lesion may be created. In keeping with the present disclosure, the resection device 1100 may be used to create the tissue core, to ligate the tissue core and to seal the tissue core and sever it from the surrounding tissue as described hereinabove. The tissue core may then be removed from the channel. As an example, a cavity port may be inserted in the channel to facilitate subsequent treatment of the target lesion site through chemotherapy and/or energy-based tumor extirpation such as radiation. As a further example, a cavity port may be disposed on the perimeter of the tissue resection apparatus. When the apparatus is removed from the tissue site, the cavity port may remain in place or may be removed.

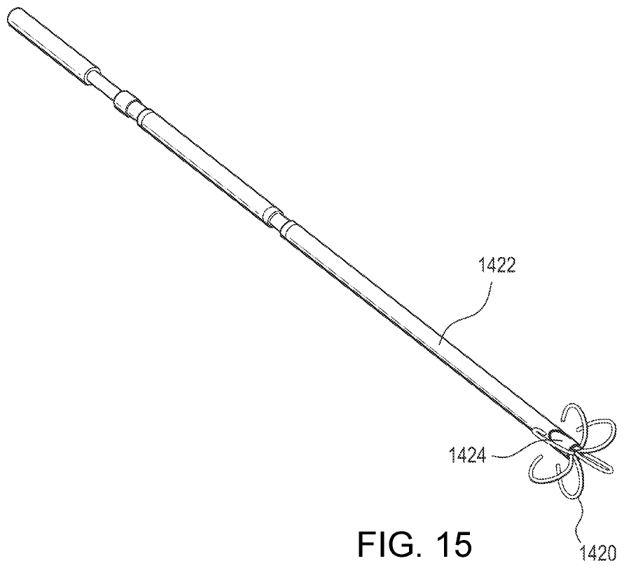

The anchor depicted in FIG. 15 may be suitable for use in performing the method for removing tissue lesions described herein. The anchor may comprise an outer tube 1422 having a sufficiently sharp edge to pierce the chest cavity tissue and lung without causing excess damage and an inner tube 1424 disposed within the outer tube 1422. One or more tines or fingers 1426 may be formed or preformed from shape memory material, e.g., Nitinol, and attached to the end of the inner tube 1424. The outer tube 1422 may be retractably disposed over the inner tube 1424 such that when the outer tube 1422 is retracted, the tines 1426 assume their preformed shape as shown. In keeping with the present disclosure, the outer tube 1422 may be retracted after it has pierced the lung lesion thereby causing the tines 1426 to engage the lung lesion. Other suitable anchors may include coils and suction-based structures.

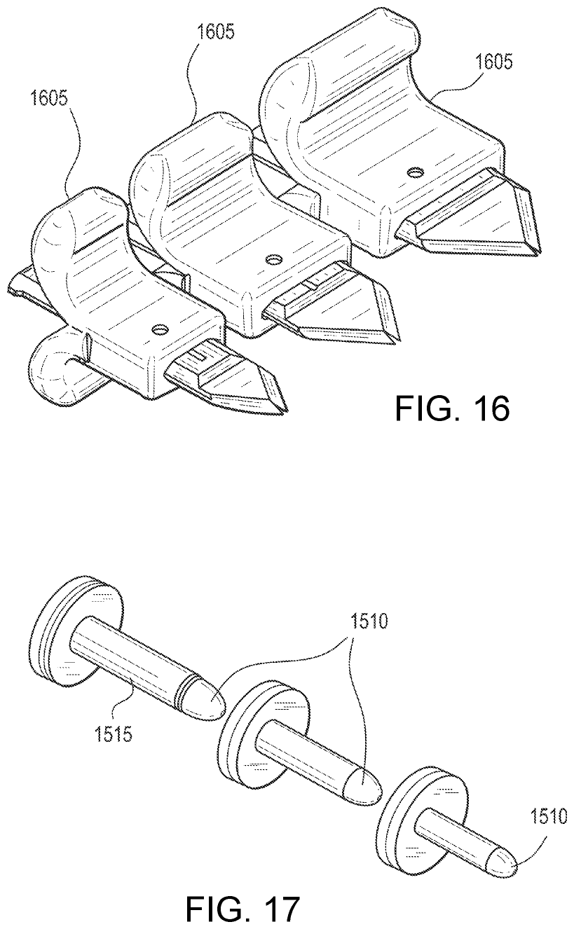

The incision blades depicted in FIG. 16 may be suitable for use in performing the method for removing tissue lesions described herein. Once the anchor 1400 is set, it may be preferable to create a small cut or incision to facilitate insertion of chest wall tissue dilator. Incision blades 1605 may be used to make a wider cut. The incision blades 1605 may successive. The incision blades 1605 may include a central aperture which may allow them to be coaxially advanced along the anchor needle 1405 to create a wider cut in the chest wall, with each successive blade being larger than the previous blade, thereby increasing the width of the incision.

The tissue dilator depicted in FIG. 17 may be suitable for use in performing the method for removing tissue lesions described herein. The tissue dilator may comprise any suitable device for creating a channel in organic tissue. In one exemplary embodiment, the tissue dilator assembly includes a single cylindrical rod with a rounded end 1510 or a cylindrical rod with a rounded end and a rigid sleeve arrangement 1515. Successive tissue dilators may be coaxially advanced along the anchor needle to create tissue tract or channel in the chest wall, with each successive dilator being larger than the previous dilator, thereby increasing the diameter of the channel. Once a final dilator with rigid sleeve is deployed, the inner rod 1505 may be removed, leaving the rigid sleeve in the intercoastal space between ribs to create direct passage to the lung pleura.

Any tissue resection device capable of penetrating lung tissue and creating a tissue core including a target lesion may be suitable for use in performing the method for removing tissue lesions described herein. The tissue resection device 1100 described hereinbefore is preferred.

Once the tissue resection device 1100 is removed, a small channel in the lung may exist where the target lesion was removed. This channel may be utilized to introduce an energy-based ablation device and/or localized chemotherapy depending on the results of the tissue diagnosis. Accordingly, the method and system of the present disclosure may not only be utilized to ensure an effective biopsy is performed, but also complete removal of the lesion with minimal healthy lung tissue removal is accomplished.

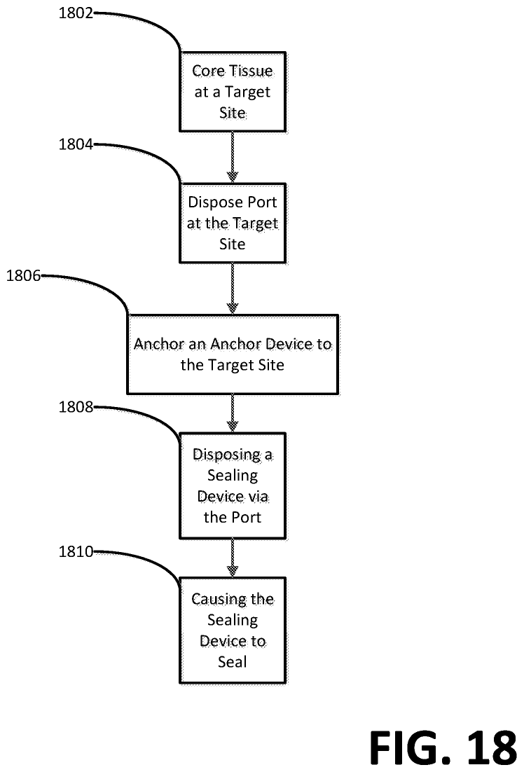

FIG. 18 shows a flow diagram of an example method. At 1802, tissue at a target site may be cored such that a tissue core is removed from the target site thereby creating a core cavity at the target site. Coring tissue at a target site may comprise transecting and sealing tissue. Coring tissue at a target site may comprise disposing a tissue coring apparatus adjacent to a target tissue site. The tissue coring apparatus may comprise a first clamping element comprising a helical coil, a second clamping element, the second clamping element being positioned to oppose at least a portion of the first clamping element, a first and second electrode configured for the delivery of radiofrequency energy for sealing tissue, and/or a cutting element configured for the transection of at least a portion of the sealed tissue. Other apparatus may be used.

At 1804, a port may be disposed to provide access to a target site. A port (e.g., chest port 102 (FIG. 1)) may be disposed to provide access to a target site. A user may dispose a port to provide access to a target site. The target site may comprise biological tissue. The target site may comprise tissue of a lung. The target site may comprise a cored tissue. The target site may comprise a punctured tissue. The target site may comprise at least a portion of a lung.

At 1806, an anchor device may be anchored to a surface at the target site. As an example, a user may anchor the anchor device, via the port, to a surface at the target site.

At 1808, a sealing device may be disposed adjacent the target site, for example via the port. The sealing device may comprise an inflatable balloon. The sealing device may comprise an inflatable balloon catheter. The lung may be caused to collapse prior to disposing the sealing device adjacent the target site. The lung may be allowed to ventilate while the sealing device is sealing the target site.

At 1810, the sealing device may be caused to seal the target site. Causing the sealing device to seal the target site may comprise causing at least a portion of the sealing device to abut a portion of the target site.

The present disclosure comprises at least the following aspects:

Aspect 1. A method for sealing tissue at a cored site, the method comprising: coring tissue at a target site such that a tissue core is removed from the target site thereby creating a core cavity at the target site; disposing a sealing device adjacent the target site; causing the sealing device to seal at least a portion of the core cavity at the target site; and spacing the sealing device from the target site.

Aspect 2. The method of aspect 1 wherein coring tissue at a target site comprises cauterizing and cutting tissue.

Aspect 3. The method of any one of aspects 1-2, wherein coring tissue at a target site comprises first sealing blood vessels then slicing tissue to form the tissue core.

Aspect 4. The method of any one of aspects 1-3, wherein the coring tissue comprises delivery of radiofrequency energy.

Aspect 5. The method of any one of aspects 1-4, wherein the coring tissue comprises mechanical transection.

Aspect 6. The method of any one of aspects 1-5, wherein the coring tissue comprises mechanical compression and delivery of radiofrequency energy.

Aspect 7. The method of any one of aspects 1-6, wherein the coring tissue comprises mechanical compression, delivery of radiofrequency energy, and mechanical transection.

Aspect 8. The method of any one of aspects 1-7, wherein the coring tissue comprises delivery of radiofrequency energy and mechanical transection.

Aspect 9. The method of any one of aspects 1-8, wherein the coring tissue comprises transection with an energized wire.

Aspect 10. The method of any one of aspects 1-9, wherein the target site comprises tissue of a lung.

Aspect 11. The method of any one of aspects 1-10, wherein the sealing device comprises an inflatable balloon.

Aspect 12. The method of any one of aspects 1-11, wherein the sealing device comprises an inflatable balloon catheter.

Aspect 13. The method of any one of aspects 1-11, wherein the inflatable balloon is configured for delivery of radiofrequency energy to seal tissue.

Aspect 14. The method of any one of aspects 1-11, wherein the inflatable balloon is configured for delivery of thermal energy to seal tissue.

Aspect 15. The method of any one of aspects 1-11, wherein the inflatable balloon is configured to apply mechanical compression to seal tissue.

Aspect 16. The method of any one of aspects 1-11, wherein the inflatable balloon is configured for delivery of a hemostatic adjunct.

Aspect 17. The method of any one of aspects 1-11, wherein the inflatable balloon is configured delivery of a biocompatible tissue adhesive.

Aspect 18. The method of any one of aspects 1-11, wherein the inflatable balloon comprises a bio-absorbable material.

Aspect 19. The method of any one of aspects 1-18, wherein the sealing device comprises a probe configured for delivery of microwave radiation.

Aspect 20. The method of any one of aspects 1-19, wherein causing the sealing device to seal at least a portion of the core cavity at the target site comprises causing at least a portion of the sealing device to abut a wall defining the core cavity.

Aspect 21. The method of any one of aspects 1-20, wherein causing the sealing device to seal at least a portion of the core cavity at the target site comprises ablating a wall defining the core cavity.

Aspect 22. The method of any one of aspects 1-21, wherein causing the sealing device to seal at least a portion of the core cavity at the target site comprises applying pressure to a wall defining the core cavity.

Aspect 23. The method of any one of aspects 1-22, wherein the target site comprises at least a portion of a lung and further comprising causing the lung to collapse prior to disposing the sealing device adjacent the target site.

Aspect 24. The method of any one of aspects 1-23, wherein the target site comprises at least a portion of a lung and further comprising allowing the lung to ventilate while the sealing device is sealing the target site.

Aspect 25. The method of any one of aspects 1-24, further comprising disposing an anchor at the target site, wherein the disposing the sealing device adjacent the target site is implemented using the anchor.

Aspect 26. The method of any one of aspects 1-25, further comprising disposing a port adjacent the target site to provide access to the target site, wherein the disposing the sealing device adjacent the target site is implemented via the port.

Aspect 27. A method for sealing tissue at a cored site, the method comprising: coring tissue at a target site such that a tissue core is removed from the target site thereby creating a core cavity at the target site; and sealing at least a portion of the core cavity at the target site.

Aspect 28. The method of aspect 27, wherein coring tissue at a target site comprises cauterizing and cutting tissue.

Aspect 29. The method of any one of aspects 27-28, wherein coring tissue at a target site comprises first sealing blood vessels then slicing tissue to form the tissue core.

Aspect 30. The method of any one of aspects 27-29, wherein the coring tissue comprises delivery of radiofrequency energy.

Aspect 31. The method of any one of aspects 27-30, wherein the coring tissue comprises mechanical transection.

Aspect 32. The method of any one of aspects 27-31, wherein the coring tissue comprises mechanical compression and delivery of radiofrequency energy.

Aspect 33. The method of any one of aspects 27-32, wherein the coring tissue comprises mechanical compression, delivery of radiofrequency energy, and mechanical transection.

Aspect 34. The method of any one of aspects 27-33, wherein the coring tissue comprises delivery of radiofrequency energy and mechanical transection.

Aspect 35. The method of any one of aspects 27-34, wherein the coring tissue comprises transection with an energized wire.

Aspect 36. The method of any one of aspects 27-35, wherein the coring and sealing are implemented using the same device.

Aspect 37. The method of any one of aspects 27-36, wherein the target site comprises tissue of a lung.

Aspect 38. The method of any one of aspects 27-37, wherein the sealing is implemented using an inflatable balloon.

Aspect 39. The method of aspect 38, wherein the inflatable balloon is configured for delivery of radiofrequency energy to seal tissue.

Aspect 40. The method of any one of aspects 27-38, wherein the inflatable balloon is configured for delivery of thermal energy to seal tissue.

Aspect 41. The method of any one of aspects 27-38, wherein the inflatable balloon is configured to apply mechanical compression to seal tissue.

Aspect 42. The method of any one of aspects 27-38, wherein the inflatable balloon is configured for delivery of a hemostatic adjunct.

Aspect 43. The method of any one of aspects 27-38, wherein the inflatable balloon is configured delivery of a biocompatible tissue adhesive.

Aspect 44. The method of any one of aspects 27-38, wherein the inflatable balloon comprises a bio-absorbable material.

Aspect 45. The method of any one of aspects 27-44, wherein the sealing is implemented using a probe configured for delivery of microwave radiation.

Aspect 46. The method of any one of aspects 27-45, wherein sealing comprises causing at least a portion of a sealing device to abut a wall defining the core cavity.

Aspect 47. The method of any one of aspects 27-46, wherein sealing comprises ablating a wall defining the core cavity.

Aspect 48. The method of any one of aspects 27-47, wherein sealing comprises applying pressure to a wall defining the core cavity.

Aspect 49. The method of any one of aspects 27-48, wherein sealing comprises causing the lung to collapse prior to sealing the target site.

Aspect 50. The method of any one of aspects 27-49, wherein sealing comprises allowing the lung to ventilate while sealing the target site.

Aspect 51. The method of any one of aspects 27-50, wherein the sealing is implemented using a sealing device and further comprising spacing the sealing device from the target site after sealing.

Aspect 52. The method of any one of aspects 27-51, wherein the sealing is implemented using a sealing device and further comprising leaving the sealing device at the target site after sealing.

Aspect 53. The method of aspect 52, wherein the sealing device is implantable or absorbable.

Aspect 54. A method for sealing tissue at a cored site, the method comprising: coring tissue at a target site such that a tissue core is removed from the target site thereby creating a core cavity at the target site; disposing a port adjacent the target site to provide access to the core cavity; anchoring an anchor device to the target site; disposing, via the port and using the anchor device, a sealing device adjacent the target site; and causing the sealing device to seal at least a portion of the core cavity at the target site.

Aspect 55. The method of aspect 54, wherein coring tissue at a target site comprises cauterizing and cutting tissue.

Aspect 56. The method of any one of aspects 54-55, wherein coring tissue at a target site comprises first sealing blood vessels then slicing tissue to form the tissue core.

Aspect 57. The method of any one of aspects 54-56, wherein the coring tissue comprises delivery of radiofrequency energy.

Aspect 58. The method of any one of aspects 54-57, wherein the coring tissue comprises mechanical transection.

Aspect 59. The method of any one of aspects 54-58, wherein the coring tissue comprises mechanical compression and delivery of radiofrequency energy.

Aspect 60. The method of any one of aspects 54-59, wherein the coring tissue comprises mechanical compression, delivery of radiofrequency energy, and mechanical transection.

Aspect 61. The method of any one of aspects 54-60, wherein the coring tissue comprises delivery of radiofrequency energy and mechanical transection.

Aspect 62. The method of any one of aspects 54-61, wherein the coring tissue comprises transection with an energized wire.

Aspect 63. The method of any one of aspects 54-62, wherein the target site comprises tissue of a lung.

Aspect 64. The method of any one of aspects 54-63, wherein the sealing device comprises an inflatable balloon.

Aspect 65. The method of aspect 64, wherein the sealing device comprises an inflatable balloon catheter.

Aspect 66. The method of any one of aspects 54-64, wherein the inflatable balloon is configured for delivery of radiofrequency energy to seal tissue.

Aspect 67. The method of any one of aspects 54-64, wherein the inflatable balloon is configured for delivery of thermal energy to seal tissue.

Aspect 68. The method of any one of aspects 54-64, wherein the inflatable balloon is configured to apply mechanical compression to seal tissue.

Aspect 69. The method of any one of aspects 54-64, wherein the inflatable balloon is configured for delivery of a hemostatic adjunct.

Aspect 70. The method of any one of aspects 54-64, wherein the inflatable balloon is configured delivery of a biocompatible tissue adhesive.

Aspect 71. The method of any one of aspects 54-64, wherein the inflatable balloon comprises a bio-absorbable material.

Aspect 72. The method of any one of aspects 54-71, wherein the sealing device comprises a probe configured for delivery of microwave radiation.

Aspect 73. The method of any one of aspects 54-72, wherein causing the sealing device to seal at least a portion of the core cavity at the target site comprises causing at least a portion of the sealing device to abut a wall defining the core cavity.

Aspect 74. The method of any one of aspects 54-73, wherein causing the sealing device to seal at least a portion of the core cavity at the target site comprises ablating a wall defining the core cavity.

Aspect 75. The method of any one of aspects 54-74, wherein causing the sealing device to seal at least a portion of the core cavity at the target site comprises applying pressure to a wall defining the core cavity.

Aspect 76. The method of any one of aspects 54-75, wherein the target site comprises at least a portion of a lung and further comprising causing the lung to collapse prior to disposing the sealing device adjacent the target site.

Aspect 77. The method of any one of aspects 54-76, wherein the target site comprises at least a portion of a lung and further comprising allowing the lung to ventilate while the sealing device is sealing the target site.

Aspect 78. The method of any one of aspects 54-77, further comprising spacing the sealing device from the target site after sealing.

Aspect 79. The method of any one of aspects 54-78, further comprising leaving the sealing device at the target site after sealing.

Aspect 80. The method of aspect 79, wherein the sealing device is implantable or absorbable.

Although shown and described is what is believed to be the most practical and preferred embodiments, it is apparent that departures from specific designs and methods described and shown will suggest themselves to those skilled in the art and may be used without departing from the spirit and scope of the invention. For example, the systems, devices and methods described herein for removal of lesions from the lung. It will be appreciated by the skilled artisan that the devices and methods described herein may are not limited to the lung and could be used for tissue resection and lesion removal in other areas of the body. The present invention is not restricted to the particular constructions described and illustrated, but should be constructed to cohere with all modifications that may fall within the scope of the appended claims.

What is claimed is:

1. A method for sealing tissue at a cored site, the method comprising:
coring tissue at a target site such that a tissue core is removed from the target site thereby creating a core cavity at the target site;
disposing a sealing device adjacent the target site;
causing the sealing device to seal at least a portion of the core cavity at the target site; and
spacing the sealing device from the target site.

2. The method of claim 1, wherein coring tissue at a target site comprises one or more of cauterizing and cutting tissue, sealing blood vessels then slicing tissue to form the tissue core, delivery of radiofrequency energy, mechanical transection, mechanical compression, or transection with an energized wire.

3. The method of claim 1, wherein the sealing device comprises an inflatable balloon.

4. The method of claim 3, wherein the inflatable balloon is configured for one or more of delivery of radiofrequency energy to seal tissue, delivery of thermal energy to seal tissue, applying mechanical compression to seal tissue, delivery of a hemostatic adjunct, or delivery of a biocompatible tissue adhesive.

5. The method of claim 1, wherein the sealing device comprises one or more of a bio-absorbable material, or a probe configured for delivery of microwave radiation.

6. The method of claim 1, wherein causing the sealing device to seal at least a portion of the core cavity at the target site comprises one or more of: causing at least a portion of the sealing device to abut a wall defining the core cavity; ablating a wall defining the core cavity; or applying pressure to a wall defining the core cavity.

7. The method of claim 1, wherein the target site comprises at least a portion of a lung and further comprising causing the lung to collapse prior to disposing the sealing device adjacent the target site.

8. The method of claim 1, wherein the target site comprises at least a portion of a lung and further comprising allowing the lung to ventilate while the sealing device is sealing the target site.

9. The method of claim 1, further comprising disposing an anchor at the target site, wherein the disposing the sealing device adjacent the target site is implemented using the anchor.

10. The method of claim 1, further comprising disposing a port adjacent the target site to provide access to the target site, wherein the disposing the sealing device adjacent the target site is implemented via the port.

11. A method for sealing tissue at a cored site, the method comprising:
coring tissue at a target site such that a tissue core is removed from the target site thereby creating a core cavity at the target site; and
sealing at least a portion of the core cavity at the target site.

12. The method of claim 11, wherein coring tissue at a target site comprises one or more of cauterizing and cutting tissue, sealing blood vessels then slicing tissue to form the tissue core, delivery of radiofrequency energy, mechanical transection, mechanical compression, or transection with an energized wire.

13. The method of claim 11, wherein the sealing is effected using a sealing device comprising one or more of an inflatable balloon, a bio-absorbable material, or a probe configured for delivery of microwave radiation.

14. The method of claim 11, wherein sealing at least a portion of the core cavity at the target site comprises one or more of: causing at least a portion of a sealing device to abut a wall defining the core cavity; ablating a wall defining the core cavity; or applying pressure to a wall defining the core cavity.

15. The method of claim 11, wherein the target site comprises at least a portion of a lung and further comprising causing the lung to collapse prior to sealing.

16. The method of claim 11, wherein the target site comprises at least a portion of a lung and further comprising allowing the lung to ventilate while sealing.

17. The method of claim 11, wherein the sealing is implemented using a sealing device and further comprising spacing the sealing device from the target site after sealing.

18. The method of claim 11, wherein the sealing is implemented using a sealing device and further comprising leaving the sealing device at the target site after sealing.

19. The method of claim 11, wherein the sealing is implemented using a sealing device and wherein the sealing device is implantable or absorbable.

20. A method for sealing tissue at a cored site, the method comprising:
coring tissue at a target site such that a tissue core is removed from the target site thereby creating a core cavity at the target site;
disposing a port adjacent the target site to provide access to the core cavity;
anchoring an anchor device to the target site;
disposing, via the port and using the anchor device, a sealing device adjacent the target site; and
causing the sealing device to seal at least a portion of the core cavity at the target site.
